(19) United States
(12) Patent Application Publication
(10) Pub. No.: US 2009/0311224 A1
(43) Pub. Date: Dec. 17, 2009

(54) THERAPEUTIC AGENT COMPRISING LIPOCALIN 2 AGAINST CANCER METASTASIS, AND METHODS OF EARLY DIAGNOSIS AND INHIBITION OF CANCER METASTASIS USING LIPOCALIN 2

(76) Inventors: Eun-kyoung Lee, Seoul (KR); Ho-jeong Lee, Kyonggi-do (KR); Kong-ju Lee, Kyonggi-do (KR); Jang-seong Kim, Suwon-si (KE); Doo-bong Park, Seoul (KR); Yeup Yoon, Kwacheon-si (KR); Hyun-jun Kim, Yongin-si (KR); In-hwan Lim, Yongin-si (KR)

Correspondence Address: JHK LAW, P.O. BOX 1078, LA CANADA, CA, 91012-1078, US

(21) Appl. No.: 11/815,088

(22) PCT Filed: Feb. 24, 2006
(86) PCT No.: PCT/KR06/00640

§ 371 Date: Jul. 30, 2007

(30) Foreign Application Priority Data
Feb. 28, 2005 (KR) 10-2005-0016514

(51) Int. Cl. A61K 35/12; C07H 21/00; C12N 15/63; C12N 5/00; A61K 31/7052; C07K 14/00; A61K 38/16; C07K 16/00; A61P 35/00; C12Q 1/68

(52) U.S. Cl. 424/93.21; 536/23.5; 435/320.1; 435/325; 514/44 R; 530/350; 514/12; 530/389.1; 536/24.33; 530/388.1; 435/6

(57) ABSTRACT

The present invention relates to a pharmaceutical composition for the inhibition of cancer metastasis, more precisely, a novel pharmaceutical composition against cancer metastasis comprising lipocalin 2 protein, a gene encoding the protein, an expression vector containing the gene or cells transfected with the expression vector as an effective ingredient, a method for the inhibition of cancer metastasis using the composition, a diagnostic kit for the prediction of cancer metastasis, a method for the selection of a metastasis risk group using the kit, a novel pharmaceutical composition for the inhibition of cancer growth and a method for the inhibition of cancer growth using the same. The pharmaceutical composition of the present invention specifically inhibits cancer metastasis, so that it can improve the effect of cancer treatment dramatically. And, the diagnostic kit and the method for the selection of a metastasis risk group using the kit enable the selection of a metastasis risk group by measuring the level of lipocalin 2 in tumor tissues or in body fluid. Therefore, the kit and the method can contribute to the effective clinical control of a cancer patient. Further, the composition of the invention can be effectively used for the treatment of liver cancer owing to its liver cancer growth inhibitory effect.

### TECHNICAL FIELD

The present invention relates to a pharmaceutical composition against cancer metastasis, more precisely, a pharmaceutical composition against cancer metastasis containing human lipocalin 2 as an effective ingredient and methods of diagnosis and inhibition of cancer metastasis using the same.

### BACKGROUND ART

After all the treatments for cancer including surgical operation, chemotherapy and radiotherapy, cancer is still lethal to patients owing to its metastasis. The primary tumor patients who are in the early stage of cancer before metastasis have high chance of recovery. However, early diagnosis itself is not easy and in fact in most cases, metastasis is already begun when the primary tumor is found. In general, metastasis is multicentric and systemic. And the judgment of metastasis itself is difficult, making the current cancer-treatment unsatisfactory. But, metastasis is an inefficient process, that is, only a minute part of a tumor can be turned into a metastatic cancer after being through many steps of metastasis. Therefore, to identify a clinically and biologically useful target and to develop a method of inhibiting the target and an effective therapeutic method which can remarkably reduce the death of cancer patients caused by metastasis, the entire procedure of metastasis should be well understood.

Metastasis is a complex process comprising the following steps: angiogenesis-dependent growth of a primary tumor; invasion of metastatic cancer cells into blood vessels and lymphatic vessels from the primary tumor (intravasation); survival of the cancer cells in the blood vessels; translocation and invasion of the cancer cells to distant organs or tissues; and the growth of a newly generated micrometastasis to a macroscopic metastasis through angiogenesis (Chambers et al., Nat. Rev. Cancer, 2: 563-572, 2002). Theoretically, the inhibition of any step of those above can successfully interrupt the overall metastasis process.

Distant metastasis is not always detected in every patient with a primary tumor. The finding that a primary tumor is necessary but not sufficient for distant metastasis suggests a possibility that the loss of function of a specific gene(s) may inhibit the metastasis of the tumor cells. Based on this hypothesis, at least 11 metastasis suppressor genes have been identified to date (Kauffman et al., J. Urol., 169: 1122-1133, 2003; Shevde et al., Cancer Lett., 198: 1-20, 2003; Berger et al., Anticancer Drugs, 15: 559-568, 2004). In contrast to the tumor suppressor gene which is characterized by inducing tumor growth by the functional loss of it, the metastasis suppressor gene is defined as a gene that does not affect the growth of a primary tumor but selectively inhibits distant metastasis. Thus, a metastasis suppressor gene can be effectively used for the pathological discrimination between malignant tumors and indolent tissues, as well as the designing of a tailored-treatment strategy fitted for each individual patient by selecting a group of patients with a high risk of metastasis in the future.

The details of the mechanism by which metastasis suppressor genes inhibit the metastasis have not fully elucidated yet. According to the previous reports, even a tumor cell that is separated from a primary cancer and invades successfully into a distant tissue, occasionally fails in proliferation in the distant site (Chambers et al., Nat. Rev. Cancer, 2: 563-572, 2002; Yoshida et al., J. Natl. Cancer Inst., 92: 1717-1730, 2000). Such finding implies that a cancer cell which has invaded into a distant tissue through blood vessels from a primary cancer is under post-extravasation growth control during metastatic colonization, and this might be a critical rate-limiting step for the completion of metastasis. Although several signal transduction pathways have been reported to be affected by some metastasis suppressor proteins (Shevde et al., Cancer Lett., 198: 1-20, 2003; Kauffman et al., J. Urol., 169: 1122-1133, 2003), for most metastasis suppressor proteins, the mechanisms of their regulation of meatastatic colonization are not well understood. Metastasis is a very complicated process resulted from various genetic or epigenetic mutations and each stage of metastasis is believed to be regulated by a specific intracellular signal transduction pathway or integration of the various signal transduction pathways. The up- or down-regulation of a specific gene in the metastasis-associated signal transduction pathway will inevitably have broader effects than intended, because each pathway may affect the regulation of as yet undocumented process. Therefore, metastasis suppression by a specific metastasis suppressor gene should be considered in terms of the signal transduction pathways in which it participate, rather than the increase or decrease of the expression of the gene itself (Griend et al., J. Natl. Cancer Inst., 96: 344-345, 2004). The proliferation of a cancer cell (for example, colorectal cancer cell) in an organ (for example, liver) and/or a tissue depends on the specific genetic characteristics of the cancer cell including the expression of growth factor receptors such as an epidermal growth factor (EGF) receptor, a specific microenvironment of the tissue including the expression of a specific growth factor (for example, transforming growth factor-alpha) related to the receptors, and their interaction (Chambers et al., Nat. Rev. Cancer, 2: 563-572, 2002; Radinsky, Cancer Metastasis Rev., 14: 323-338, 1995; Fidler, Natl. Cancer Inst., 87: 1588-1592, 1995; Radinsky, Eur. J. Cancer, 31A: 1091-1095, 1995; Radinsky and Ellis, Surg. Oncol. Clin. N. Am., 5: 215-229, 1996). Thus, metastasis to a distant site makes different results according to the combination (or interaction) of the cancer cell and microenvironment in the distant site. As a result, it is impossible to generalize metastasis inhibitory effect caused by the change of a metastasis-related gene and its signal transduction pathway observed in a specific model and/or a tissue. For example, c-Jun N-terminal kinase(JNK)/p38 signal transduction pathway that is activated by MAPK kinase 4 (mitogen-activated protein kinase kinase 4, MKK4), one of metastasis suppressor genes, is closely associated with the suppression of the metastasis of prostatic cancer and ovarian cancer (Yamada et al., Cancer Res., 62: 6717-6723, 2002; Kim et al., Cancer Res., 61: 2833-2837, 2001), whereas in non-small cell lung carcinomas, the same signal transduction pathway activated by MKK4 was reported to play an important role in the progression to a malignant tumor (Xiao et al., Cancer Res., 60: 400-408, 2000). Moreover, the up-regulation of connective tissue growth factor (CTGF) is closely associated to the decrease of disease-free survival in breast cancer, pancreatic cancer and skin cancer patients but at the same time inhibits the metastasis of lung cancer (Chang et al., J. Natl. Cancer Inst., 96: 364-375, 2004; Xie et al., Cancer Res., 61: 8917-8923, 2001; Wenger et al., Oncogene, 18: 1073-1080, 1999; Kubo et al., Br. J. Dermatol., 139: 192-197, 1998). Therefore, metastasis suppressing effect of a specific metastasis suppressor gene and a signal transduction pathway mediated by the gene has to be determined in a case-by-case basis by the types of cancer and the site of metastasis.

Lipocalins are an evolutionarily well-conserved family of proteins. Despite the low degree of overall amino acid identity, the lipocalins share a common tertiary structure consisting of an eight-stranded anti-parallel beta-sheet surrounding a cup-shaped ligand-binding pocket. Lipocalins are characterized by several common characteristics including the binding with a variety of hydrophobic molecules and cell surface receptors and the formation a complex with soluble macromolecules. The major function of lipocalins has been known to the transport of small hydrophobic ligands. However, according to recent reports, it is believed to have much more functions including retinol transport, coloration, olfaction, peromon transport and biosynthesis of prostaglandins, etc. In addition, lipocalins regulates immune responses and homeostasis in cells (Flower, D. R., Biochem. J., 318: 1-14, 1996).

Lipocalin 2 (LCN2) or neutrophil gelatinase-associated lipocalin (NGAL) is an approximately 25 kDa glycoprotein, which was initially purified from secretory granules of neutrophils (Kjeldsen et al., J. Biol. Chem., 268: 10425-10432, 1993; Triebel et al., FEBS Lett., 314: 386-388, 1992). Lipocalin 2 has been reported to have functions such as transport of fatty acids and iron (Chu et al., J. Pept. Res., 52: 390-397, 1998; Yang et al., Mol. Cell, 10: 1045-1056, 2002), induction of apoptosis in neutrophils and other granulocytes (Devireddy et al., Science, 293: 829-834, 2001) and inhibition of bacterial growth by binding with catecholate-type ferric siderophore which leads to iron sequestration (Goetz et al., Mol. Cell, 10: 1033-1043, 2002; Flo et al., Nature, 432: 917-921, 2004). In addition, lipocalin 2 has been suggested to act as a modulator of inflammatory responses since lipocalin 2 is up-regulated in tissues that may be exposed to microorganisms (Cowland and Borregaard, Genomics, 45: 17-23, 1997; Friedl et al., Histochem. J., 31: 433-441, 1999). It is induced by bacterial lipopolysaccharide in murine macrophages (Meheus et al., J. Immunol., 151: 1535-1547, 1993), it can bind FMLP (N-FORMYL-Met-Leu-Phe) and other lipophilic inflammatory mediators such as platelet activating factor and leukotrien B4 (Bratt et al., Biochim. Biophys. Acta, 1472: 262-269, 1999), and it is intensely synthesized in the colonic epithelium in areas of inflammation (Nielsen et al., Gut, 38: 414-420, 1996).

Lipocalin 2 has become of interest with regard to cancer since it was found lipoclinn 2 expression changes in proliferative cells. The mouse ortholog of lipoclinn 2, 24p3, is substantially induced during the transition of mouse kidney cells from a quiescent to a proliferative state as a result of SV40 and polyoma virus infection (Hraba-Renevey et al., Oncogene, 4: 601-608, 1989). Moreover, it is expressed in mouse fibroblast cells stimulated by a number of growth factors, including serum, basic fibroblast growth factor and phorbol ester (Liu Q. and Nilsen-Hamilton M., J. Biol. Chem., 270: 22565-22570, 1995). These findings suggest that NGAL may play a role in regulating cellular growth. This hypothesis is further supported by the expression of NGAL in the inflamed colon, whose epithelium is rapidly turned over, as well as in various malignant tumors (Missiaglia et al., Int. J. Cancer, 112: 100-112, 2004; Santin et al., Int. J. Cancer, 112: 14-25, 2004; Nielsen et al., Gut, 38: 414-420, 1996; Bartsch et al., FEBS Lett., 357: 255-259, 1995; Furutani et al., Cancer Lett., 122: 209-214, 1998). In human breast cancers, the expression of lipocalin 2 is significantly associated with the fraction of cells in the S-phase of cell cycle showing DNA replication (Stoesz et al., Int. J. Cancer, 79: 565-572, 1998). However, experimental evidence showing a clear causal relationship between lipocalin 2 expression and the proliferation of tumor cells is lacking.

In humans, lipocalin 2 has been identified in colonic epithelial cells in diverse inflammatory conditions including appendicitis, inflammatory bowel diseases, and colon cancers, whereas the unaffected colon displayed no or very weak lipocalin 2 expression. According to Nielson et al, lipocalin 2 was observed in the most superficial part of the cancer and no expression was found in the lymph node metastases from primary colon tumors expressing lipocalin 2 (Nielsen et al., Gut, 38: 414-420, 1996). Based on these findings, lipocalin 2 expression was thought not to be inherent in neoplastic cells; rather, it may be the result of the accompanying inflammatory reaction. However, there has been no direct experimental evidence showing the role of lipocalin 2 in the inhibition of metastasis.

### DISCLOSURE

### Technical Problem

It is an object of the present invention to provide a pharmaceutical composition for suppressing cancer metastasis or tumor growth and methods for suppressing cancer metastasis or tumor growth using the same.

It is another object of the present invention to provide a diagnostic kit that is able to predict the risk of metastasis in the future in a cancer patient and to provide a method for the prediction, by using lipocalin 2 as a cancer metastasis marker.

### Technical Solution

The present invention provides a pharmaceutical composition against metastasis comprising lipocalin 2 protein, a gene encoding the protein, an expression vector containing the gene or mammalian cells transfected with the expression vector as effective ingredients. Herein, lipocalin 2 protein is not limited to a specific one, but a whole lipocalin 2 amino acid sequence represented by SEQ. ID. No 2 or a mature form of human lipocalin 2 protein with the deletion of secretory sequence (1^{st}-20^{th} amino acid of the sequence represented by SEQ. ID. No 2) represented by SEQ. ID. No 11 is preferred, and a mature form of human lipocalin 2 protein represented by SEQ. ID. No 11 is more preferred. The gene is not limited to a specific one, but a gene represented by SEQ. ID. No 1 encoding human lipocalin 2 protein represented by SEQ. ID. No 2 or No 11 is preferred. The expression vector herein is not limited to a specific one, either, but a non-viral vector or a viral vector is preferred. At this time, the viral vector is preferably one of adenovirus vector, retrovirus vector including lentivirus vector, adeno-associated virus vector or herpes simplex virus vector. Among them, lentivirus vector is more preferred and lentivirus vector presented by pLenti-NGAL of FIG. 2 is most preferred. The pharmaceutical composition of the present invention is not always limited thereto, and can additionally contain a pharmaceutically acceptable carrier. In the meantime, the cancer herein is not limited to a specific cancer but colorectal cancer or liver cancer is preferred as an example.

The pharmaceutical composition of the present invention does not affect cancer cell growth in a colorectal cancer patient but specifically inhibits metastasis. Therefore, when it is used together with another anticancer agent, it can remarkably improve the effect of cancer therapy.

The present invention provides a pharmaceutical composition against cancer cell growth comprising lipocalin 2 protein, a gene encoding the protein, an expression vector containing the gene or mammalian cells transfected with the expression vector as an effective ingredient. Herein, lipocalin 2 protein is not limited to a specific one, but a whole lipocalin 2 amino acid sequence represented by SEQ. ID. No 2 or a mature form of human lipocalin 2 protein with the deletion of secretory sequence (1^{st}-20^{th} amino acid of the sequence represented by SEQ. ID. No 2) represented by SEQ. ID. No 11 is preferred, and a mature form of human lipocalin 2 protein represented by SEQ. ID. No 11 is more preferred. The gene is not limited to a specific one, but a gene represented by SEQ. ID. No 1 encoding human lipocalin 2 protein represented by SEQ. ID. No 2 or No 11 is preferred. The expression vector herein is not limited to a specific one, either, but a non-viral vector or a viral vector is preferred. At this time, the viral vector is preferably one of adenovirus vector, retrovirus vector including lentivirus vector, adeno-associate virus vector or herpes simplex virus vector. Among them, lentivirus vector is more preferred and lentivirus vector presented by pLenti-NGAL of FIG. 2 is most preferred. The pharmaceutical composition of the present invention is not always limited thereto, and can additionally contain a pharmaceutically acceptable carrier. In the meantime, the cancer herein is not limited to a specific cancer but liver cancer is preferred as an example.

The pharmaceutical composition of the present invention specifically inhibits cancer cell growth in a liver cancer patient and metastasis thereof. Therefore, when it is used together with other anticancer agents, it can remarkably improve the effect of cancer therapy.

The present invention provides an expression vector for gene therapy that expresses lipocalin 2. Herein, the expression vector is not limited to a specific one, but adenovirus vector, retrovirus vector including lentivirus vector, adeno-associate virus vector or herpes simplex virus vector is preferred. Among them, lentivirus vector is more preferred and lentivirus vector presented by pLenti-NGAL of FIG. 2 is most preferred. The present invention further provides a recombinant lentivirus prepared by transfection with the lentivirus vector.

The present invention also provides a cell line transfected with the expression vector or the recombinant lentivirus. At this time, the cell line is not limited to a specific one, but a normal cell line or a cancer cell line is preferred. And the cancer cell line is not limited, either but is preferably selected from a group consisting of KM12C, SW480, KM12SM, SW620, Chang liver, SK-Hep1 and Huh7.

The present invention also provides a method for the inhibition of cancer metastasis including the step of administering a pharmaceutical composition against metastasis of the invention to a cancer patient. Herein, the cancer is not limited to a specific one, but colorectal cancer or liver cancer is preferred.

The present invention further provides a novel therapeutic method for cancer including the step of administering a pharmaceutical composition against metastasis of the present invention and a conventional anticancer agent simultaneously or serially.

The present invention also provides a method for the inhibition of tumor growth including the step of administering a pharmaceutical composition for inhibiting tumor growth of the invention to a cancer patient. Herein, the cancer is not limited to a specific one, but liver cancer is preferred.

The present invention further provides a novel therapeutic method for cancer including the step of administering a pharmaceutical composition against metastasis of the present invention and a conventional anticancer agent simultaneously or serially.

Further, the present invention provides a kit for predicting the risk of tumor metastasis containing a lipocalin 2-specific antibody or a set of primers which are specific to a gene encoding the protein. Herein, the antibody is not limited to a specific one, but a monoclonal antibody is preferred and a human monoclonal antibody is more preferred. In the meantime, the primer sets are not specifically limited but primers represented by SEQ. ID. No 3 and SEQ. ID. No 4 are preferred.

The present invention also provides a method for selection of metastasis risk groups by using the kit for predicting the risk of metastasis, which is composed of the following steps: i) obtaining a cancer sample from a cancer patient; ii) preparing a sample for the detection of lipocalin 2 protein or mRNA encoding the protein from the cancer sample (dissection sample, lysate and total RNA); iii) detecting lipocalin 2 protein or mRNA encoding the protein from the sample; and iv) analyzing the level of the independent expression of lipocalin 2 or the combined expression level of lipocalin 2 and other metastasis suppressor genes. At this time, the step iii) can be performed by a method selected from a group consisting of in situ hybridization, immunohistochemical staining, ELISA (enzyme-linked immunosorbent assay), Western blot, Northern blot, RT-PCR and real-time RT-PCR, but not always limited thereto.

In the case of colorectal cancer, lipocalin 2 expression was increased in KM12C and SW480 cell lines that were derived from a primary tumor, whereas the expression of lipocalin 2 was reduced in KM12SM and SW480 cell lines, the cell lines of the same genetic origin as their respective prmary tumors but derived from metastatic cancers in liver and lymph node, respectively. Based on this finding, a hypothesis that lipocalin 2 has a function of inhibiting metastasis of colorectal cancer cells is suggested. To prove the hypothesis, the present inventors induced over-expression of lipocalin 2 in KM12SM having a high metastatic potential but low level of lipocalin 2 and then investigated the effect of the over-expression of lipocalin 2 on the proliferation and liver metastasis of KM12SM cell line.

To achieve the above object, the present inventors constructed a recombinant lentivirus vector (pLenti-NGAL) that was designed to express lipocalin 2 constitutively under the control of CMV promoter and a recombinant lentivirus harboring the vector. Then, transduction of KM12SM cell line was performed. The recombinant virus is not always limited to the lentivirus but in a preferable embodiment of the present invention, KM12SM cell lines transfected with a control lentivirus vector and the pLenti-LGAL vector designed to express lipocalin 2 were constructed (named respectively ‘SM-Mock’ and ‘SM-NGAL’) and used for further experiments.

In contrast to the hypothesis that lipocalin 2 might play an important role in cell-proliferation, there was no significant difference in colorectal cancer cell proliferation in vitro between lipocalin 2 over-expressing cell line group and a control group, and apoptosis was not induced, either. In addition, when tumor growth was also investigated in an experimental animal with subcutaneous xenograft, the over-expression of lipocalin 2 did not affect the colorectal cancer cell growth in vivo. On the other hand, in the case of liver cancer, the over-expression of lipocalin 2 inhibited the liver cancer cell growth, reduced the size of a solid tumor and decreased VEGF expression, indicating that lipocalin 2 inhibited the growth of liver cancer.

The present inventors found out through matrigel invasion assay that lipocalin 2 reduces invasive capacity of colorectal cancer cells through extracellular matrix. To form a distant metastasis, a cancer cell should be able to invade into blood vessel or lymph node from a primary tumor and able to invade into the distant tissue from the blood vessel or lymphatic vessel. In this process, decomposition of a basement membrane enveloping a primary tumor and blood vessel and lymphatic vessel is necessary. Since Matrigel is composed of basement membrane component-like extracellular Matrix, matrigel invasion can be used as a key index for invasive capacity. Thus, the decrease of invasive capability of colorectal cancer cells by the over-expression of lipocalin 2 leads to the decrease of metastatic potential. To confirm the hypothesis, a lipocalin 2 over-expressing colorectal cancer cell line (SM-NGAL) and a control cell line were injected in a spleen of a nude mouse to induce liver metastasis. As a result, liver metastasis was remarkably reduced in a mouse implanted with lipocalin 2 over-expressing colorectal cancer cell line, compared with a control mouse. Based on the above results, the present inventors completed this invention by confirming that lipocalin 2 does not affect colorectal cancer cell proliferation but remarkably reduces invasive potential of colorectal cancer cells, indicating that lipocalin 2 has a novel function as a metastasis suppressor by inhibiting metastasis of a colorectal cancer cell to the liver or the lymph node.

The present inventors also found out the fact though experiments using liver cancer cell lines that lipocalin 2 reduces the expression of matrix metalloprotease-2 in liver cancer cells and thereby reduces the invasive potential. The finding indicates that lipocalin 2 is acting as a metastasis suppressor not only for colorectal cancer but also for various cancers.

High level of lipocalin 2 expression is observed in a primary cancer of a colorectal cancer patient, but not detected in a metastatic colony in the liver of the same patient. The expression of lipocalin 2 has been understood as a non-genetic phenotype accompanied by inflammation in epithelial cells including colonic epithelial cells until the present invention was completed. However, the level of lipocalin 2 was in inverse proportion to metastatic potential in those cell lines which were sub-cultured serially in vivo under inflammation-free condition. This finding indicates that lipocalin 2 expression is a kind of inherent phenotype of a cancer cell. The details and reasons of different levels of lipocalin 2 in metastatic cancer and primary cancer have not been disclosed, yet. But according to recent reports, most metastasis suppressor genes do not carry mutation in a gene, unlike most tumor suppressor genes, and are regulated by epigenetic changes including DNA methylation, histone acetylation, etc. In the case of pancreatic cancer, hypermethylation of the promoter region of lipocalin 2 is observed in normal pancreatic cells, but promoter methylation was suppressed in pancreatic cancer cells having high level of lipocalin 2 (Sato et al., Cancer Res., 63: 4158-4166, 2003). The above results indicate that the lipocalin 2 expression in colorectal cancer may be controlled by the similar regulatory mechanisms.

Based on the above results, the present inventors performed Northern blotting or Western blotting to investigate the independent expression of lipocalin 2 either alone or in combination with another tumor suppressor gene in tumor tissues of a cancer patient or in cancer cells in body fluid such as blood or lymph, by which the chances of metastasis can be indirectly predicted. The method for analyzing the expression of lipocalin 2 is not limited to Northern blotting and Western blotting. For example, a primary tumor tissue can be examined by \textcircled{1} Western blot, \textcircled{2} immunohistochemical staining or \textcircled{3} in situ hybridization, and a tumor tissue or isolated cancer cells are examined by \textcircled{1} reverse transcriptase-polymerase chain reaction (RT-PCR), \textcircled{2} real-time quantitative RT-PCR or \textcircled{3} Northern blot.

Lipocalin 2 is a secreted protein by the processing of a leader peptide after being biosynthesized in a cell. The introduction of lipocalin 2 gene showed significant inhibition of colorectal cancer liver metastasis. Therefore, it is expected that the exogenous introduction of a recombinant lipocalin 2 protein might have similar metastasis inhibitory effect. In fact, the present inventors proved in preferred embodiments of the invention that a recombinant lipocalin 2 protein inhibits the invasion of KM12SM colorectal cancer cells through Matrigel. It also reduces the expression of matrix metalloproteinase-2 (MMP-2) which plays an important role in metastasis by decomposing basement membrane and lowers the matrigel invasive potential in a liver cancer cell line. It was also proved that a recombinant lipocalin 2 protein can effectively suppress liver metastasis of a colorectal cancer cell by the systemic treatment of the protein. Therefore, considering all the above results, it was confirmed that a recombinant lipocalin 2 protein can be used as a therapeutic agent against metastasis.

A pharmaceutical composition of the present invention contains the above effective ingredient by 0.0001-50 weight % for the gross weight of the composition.

The composition of the present invention can additionally include, in addition to the effective ingredient, one or more effective ingredients having the same or similar functions to the extract of the invention.

The composition of the present invention can also include, in addition to the above-mentioned effective ingredients, one or more pharmaceutically acceptable carriers for the administration. A pharmaceutically acceptable carrier can be selected or be prepared by mixing more than one ingredients selected from a group consisting of saline, sterilized water, Ringer's solution, buffered saline, dextrose solution, maltodextrose solution, glycerol, ethanol and liposome. Other general additives such as anti-oxidative agent, buffer solution, bacteriostatic agent, etc, can be added. In order to prepare injectable solutions, pills, capsules, granules, tablets, diluents, dispersing agents, surfactants, binders, or lubricants can be additionally included. The composition of the present invention can further be prepared in suitable forms for each disease or according to ingredients by following a method represented in Remington's Pharmaceutical Science (the newest edition), Mack Publishing Company, Easton Pa.

The composition of the present invention can be administered orally or parenterally (for example, intravenous, hypodermic, local or peritoneal injection). Among them, parenteral administration is preferred and intravenous injection is more preferred. The effective dosage of the composition can be determined according to weight, age, gender, health condition, diet, administration frequency, administration method, excretion and severity of a disease. The dosage of the compound is 0.1˜100 mg/kg per day, and preferably 0.5˜10 mg/kg per day. Administration frequency is once a day or preferably a few times a day.

Lipocalin 2 or lipocalin 2 expression vector of the present invention was intravenously injected into mice to investigate toxicity. As a result, it was evaluated to be a safe substance since its estimated LD₅₀ value is much greater than 1,000 mg/kg in mice.

The composition of the present invention can be administered singly or treated along with surgical operation, hormone therapy, chemotherapy and biological reaction regulator, to treat a cancer.

### [Advantageous Effects]

The present invention relates to a novel pharmaceutical composition against cancer metastasis comprising lipocalin 2 protein, a gene encoding the lipocalin 2 protein, an expression vector harboring the gene and a cell transfected with the expression vector as effective ingredients, a method for inhibiting cancer metastasis using the composition, a diagnostic kit to predict the risk of cancer metastasis and a method for the selection of a metastasis risk group. The pharmaceutical composition of the present invention improves cancer treatment effect significantly by inhibiting cancer metastasis specifically, and the diagnostic kit and the method for the selection of a metastasis risk group using the kit enable the selection of a high risk group of metastasis by measuring the level of lipocalin 2 in tumor tissues or body fluid. In addition, the composition of the invention inhibits the proliferation of liver cancer cells, the growth of a solid tumor and the expression of VEGF.

### DESCRIPTION OF DRAWINGS

The application of the preferred embodiments of the present invention is best understood with reference to the accompanying drawings, wherein:

FIG. 1 is a photograph of Northern blot analysis illustrating the lipocalin 2 expressions in colorectal cancer cells having different metastatic capacities, FIG. 2 is a schematic diagram showing the maps of pLenti-NGAL and pLenti-L6H of the present invention, FIG. 3 is a photograph of Northern blot analysis illustrating the over-expression of lipocalin 2 gene in an established lipocalin 2 over-expressing colon cancer cell line,

FIG. 4 is a photograph of Western blot analysis illustrating the over-expression of lipocalin 2 protein in concentrated culture supernatants of colon cancer cell lines confirmed to over-express lipocalin 2,

FIG. 5-FIG. 7 are a photograph of Northern blot analysis showing the over-expression of lipocalin 2 gene in established liver cancer cell lines Chang liver (FIG. 5), SK-Hep-1 (FIG. 6) and Huh-7 (FIG. 7),

FIG. 8 is a schematic diagram showing the cleavage map of the expression vector pNGAL6H for over-expression of lipocalin 2,

FIG. 9 is a photograph of polyacrylamide gel illustrating the processes of lipocalin 2 expression in E. coli and purification thereof,

FIG. 10 is a graph showing the invasiveness of human colorectal cancer cell line KM12SM when lipocalin 2 gene is over-expressed therein,

FIG. 11 is a graph showing the invasiveness of human colorectal cancer cell line KM12SM treated with a recombinant protein of lipocalin 2,

FIG. 12-FIG. 14 are graphs showing the invasive capacities of liver cancer cell lines Chang liver (FIG. 12), Huh-7 (FIG. 13) and SK-Hep-1 (FIG. 14) when lipocalin 2 protein is expressed in them,

FIG. 15 is a photograph of Northern blot analysis illustrating that MMP-2 gene expression is decreased by lipocalin 2 in Chang liver cell line,

FIG. 16 is a photograph of Northern blot analysis illustrating that MMP-2 gene expression is decreased by lipocalin 2 in SK-Hep-1 cell line,

FIG. 17 and FIG. 18 are photographs of agarose gel showing the expressions of MMP-2 gene, as assessed by RT-PCR, in liver cancer cell line Chang liver treated with the recombinant lipocalin 2 protein in a 10% FBS containing medium (FIG. 17) and in a serum-free medium (FIG. 18),

FIG. 19 is a graph showing the numbers of cells measured by cell proliferation test to investigate the effect of the over-expression of lipocalin 2 on the in vitro colorectal cancer cell growth,

FIG. 20 is a graph illustrating the tumor growth of lipocalin 2 over-expressing colorectal cancer cells and control cells that were injected subcutaneously into mice,

FIG. 21 is a photograph of Western blot analysis of lipocalin 2 proteins in the extracts of solid tumor tissues to show that lipocalin 2 is actively expressed in the subcutaneously implanted colorectal tumors in mice,

FIGS. 22, 23 and 24 are graphs showing the numbers of cells measured by cell proliferation assay to investigate the effect of the over-expression of lipocalin 2 on the in vitro growth of liver cancer cell lines Chang liver, SK-Hep1 and Huh-7,

FIG. 25 is a photograph showing the tumors extracted 47 days after hypodermic injection of control cells (SK-Mock) and lipocalin 2 over-expressing liver cancer cells (SK-NGAL; clone #3-#9) in mice, and FIG. 26 is a graph showing the volumes of the tumors,

FIG. 27 is a photograph of Northern blot analysis investigating RNAs extracted from the solid tumors, in order to determine the expressions of lipocalin 2 and VEGF (vascular endothelial cell growth factor) in SK-Mock and SK-NGAL (clone #3-#9), in which 18S ribosomal RNA is used as a loading control to make sure that equal amount of RNA is used,

FIG. 28 is a graph showing the levels of lipocalin 2 in liver cancer patients of cluster A showing poor prognosis and cluster B showing good prognosis,

FIG. 29 is a photograph of livers collected from mice 21 days after the injection of control (KM12SM, SM-Mock) and lipocalin 2 over-expressing colorectal cancer cell line SM-NGAL into the spleens of mice to induce liver metastasis,

FIG. 30 is a graph showing the number of liver metastases on the surface of the liver of FIG. 29,

FIG. 31 is a graph showing the numbers of colorectal cancer liver metastases formed on the surface of the liver measured to examine the liver metastasis inhibitory effect of the recombinant lipocalin 2 protein.

### BEST MODE

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Lipocalin 2 Expression According to the Metastatic Capacity of a Colorectal Cancer Cell

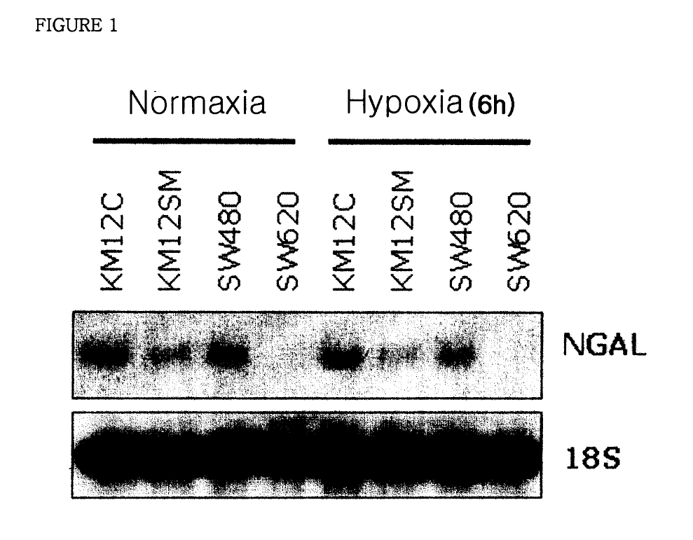

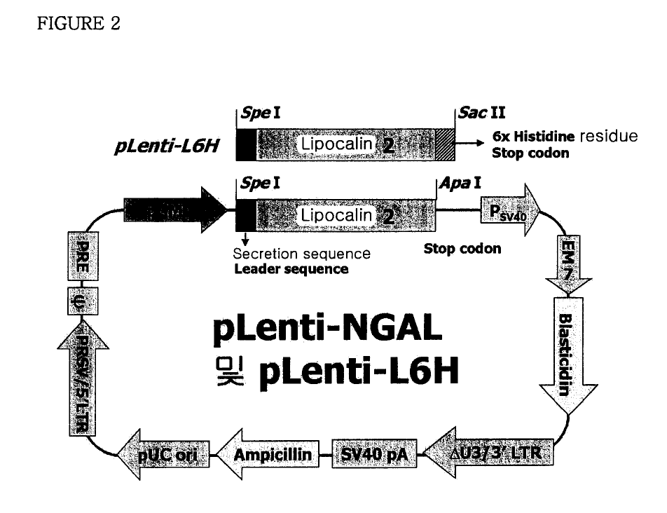

To investigate lipocalin 2 expression in relation to the metastatic capacity of a colorectal cancer cell, RNAs extracted from various colorectal cancer cell lines having a different metastatic capacity were examined by Northern blot analysis. Total RNAs were extracted by using RNA extraction reagent (Trizol, Gibco BRL, USA) from human colorectal cancer cell line KM12C purchased from Korea Cell Line Bank (Seoul, Korea), KM12SM (Morikawa et al., Cancer Res., 48: 6863-6871, 1988) that is isolated from liver metastasis colonies formed after in vivo transplantation of KM12C and has higher metastatic capacity than the mother cell line KM12C, SW480 and SW620 (Hewitt et al., J. Pathol., 192: 446-454, 2000) that is isolated from lymph node metastasis colonies of the patient from whom SW480 has been extracted and has higher metastatic capacity than the mother cell line SW480. 20 μg of the RNA was placed in 2.2 M formaldehyde denaturation gel, followed by electrophoresis. The RNA of lipocalin 2 was quantified by Northern blot using 32P-labeled lipocalin 2 DNA as a probe (FIG. 1). FIG. 1 is a photograph of Northern blot analysis illustrating the lipocalin 2 expressions in colorectal cancer cell lines having different metastatic capacities.

As shown in FIG. 1, lipocalin 2 expression was significantly higher in KM12C known to have a low metastatic capacity than in KM12SM having a rather higher metastatic capacity, which is consistent with the results in other colorectal cancer cell lines SW480 and SW620. The above results indicate that the level of lipocalin 2 is increased in cell lines having low metastatic capacities such as KM12C and SW480.

### Example 2

### Preparation of Lipocalin 2 Over-Expressing Recombinant Lentivirus

PCR (ExTaq™, TakaRa, Japan) was performed by using human liver cDNA library (Invitrogen, USA) as a template to prepare lipocalin 2 gene represented by SEQ. ID. No 1 containing its own secretory sequence. The sequences of the primers used were 5′-CACCATGCCCCTAGGTCTCCTGTGGCTG-3′ (SEQ. ID. No 3) and 5′-TCAGCCGTCGATACACTG-3′ (SEQ. ID. No 4) and the PCR was performed as follows; at 95° C. for 1 minute, at 48° C. for 1 minute and at 72° C. for 1 minute (30 cycles). Lipocalin 2 gene obtained from the PCR was cloned into pGEM-T Easy vector(Promega, USA) (pT-NGAL), and then inserted in between Spe I and Apa I restriction enzyme sites of the lentivirus expression vector (pLenti6/V5-D-TOPO, Invitrogen, USA). Particularly, restriction enzyme sites of the 5′- and 3′-ends of lipocalin 2 PCR fragment were digested with Spe I/Apa I, and the resultant lipocalin 2 gene fragment was inserted in between Spe I and Apa I sites of the lentivirus expression vector, resulting in pLenti-NGAL, a lentivirus expression vector for lipocalin 2 (FIG. 2). To construct another lentivirus expression vector for lipocalin 2 in which 6 histidines are added at 3′-end, PCR was performed with a primer sets having the sequences of 5′-ATTTAGGTGACACTATAGAATACT-3′ (SEQ. ID. No 5) and 5′-TCCCCGCGGTCAATGGTGATGGTGATGATGGCCGTCGATACACTG-3′ (SEQ. ID. No 6) respectively by using the pT-NGAL as a template. The gene fragment obtained from the PCR was treated with Spe I/Sac II and then inserted in between Spe I and Sac II sites of the lentivirus expression vector (pLenti6/V5-D-TOPO; Invitrogen, USA). The lentivirus expression vector for lipocalin 2 expression with 6 histidines at 3′-end was named pLenti-L6H (FIG. 2). pLenti-L6H was constructed only to purify the lipocalin 2 protein and the addition of 6 histidines was confirmed not to affect the functions of lipocalin 2. pLenti6/V5-D-TOPO vector without a gene insertion was used as a control vector. The nucleotide sequences of the constructed expression vectors were confirmed by DNA sequencing using primers having the sequences of 5′-CGCAAATGGGCGGTAGGCGTG-3′ (SEQ. ID. No 7) and 5′-ACCGAGGAGAGGGTTAGGGAT-3′ (SEQ. ID. No 8). The prepared lipocalin 2 expressing recombinant lentivirus expression vector (pLenti-NGAL) and a control lentivirus expression vector (pLenti6/V5-D-TOPO) were used to produce each independent recombinant lentivirus (LV-NGAL, LV-Mock) in 293FT cell line (Invitrogen, USA) (FIG. 2). FIG. 2 illustrates cleavage maps of pLenti-NGAL and pLenti-L6H. Three plasmids, pLP1, pLP2 and pLP/VSVG (ViraPower™ Lentiviral Expression System, Invitrogen, USA), the expression vectors designed to provide proteins necessary for forming lentivirus particles, were introduced into 293FT cells together with the lipocalin 2 expression vector prepared above or a control vector. 24 hours later, the medium was replaced with a fresh DMEM supplemented with 10% FBS (Gibco, USA), followed by further culture for 48-72 hours in a 37° C. 5% CO₂ incubator. Then, supernatant was obtained by centrifugation. After centrifugation (1000 rpm, 15 minutes) and filtration (0.22 μm), recombinant lentivirus solution was obtained and stored at −70° C. until use. Virus titers of the recombinant control lentivirus (LV-Mock) and lipocalin 2 expressing lentivirus (LV-NGAL) were determined by using HT1080 cell line as approximately 5×10⁵ TU (transduction unit)/ml.

### Example 3

### Establishment of Lipocalin 2 Over-Expressing Cancer Cell Line

Control and Lipocalin 2 over-expressing cell lines were constructed by using LV-Mock and LV-NGAL. Each experimental cancer cell line (colorectal cancer cell lines KM12C, SW480, KM12SM or SW620 and liver cancer cell lines Chang liver, SK-Hep1 or Huh7) was inoculated on a 6 well culture plate by 2 ml per well at the concentration of 1-2×10⁵/ml. 24 hours later, LV-Mock and LV-NGAL were added to the medium by 1.0 MOI (multiplicity of infection), leading to the transduction of cancer cells. One day later, the medium was replaced with the fresh one and then the medium was replaced with the fresh one containing 3 μg/ml of blasticidin (Invitrogen, USA) every 3-4 days. Then, recombinant cells transduced with the lentivirus were selected. After two weeks of selection, 5-10 individual clones of each of the mock cancer cell line (control) and the lipocalin 2 over-expressing cancer cell line were isolated by limiting dilution culture.

The control clones and the lipocalin 2 over-expressing clones selected for the final experiment were stored in each cell stock in liquid nitrogen tank, and the recombinant cancer cells transduced stably with the lentivirus were maintained in a medium containing 3 μg/ml of blasticidin for the further experiments. Among prepared recombinant cell lines, those cell lines obtained by transducing KM12SM with LV-Mock and LV-NGAL were named SM-Mock and SM-NGAL, respectively. The cell lines obtained by transducing Chang liver with LV-mock and LV-NGAL were named CL-Mock and CL-NGAL. Likewise, cell lines obtained by transducing Huh-7 were named H7-Mock and H7-MGAL and those obtained by transducing SK-Hep1 were named SK-Mock and SK-NGAL.

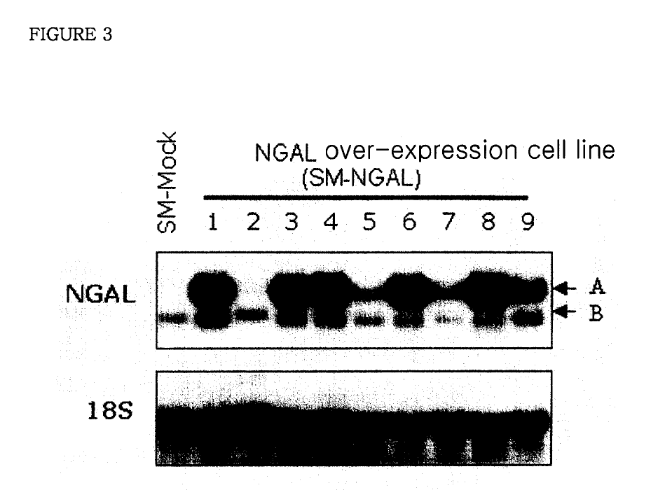

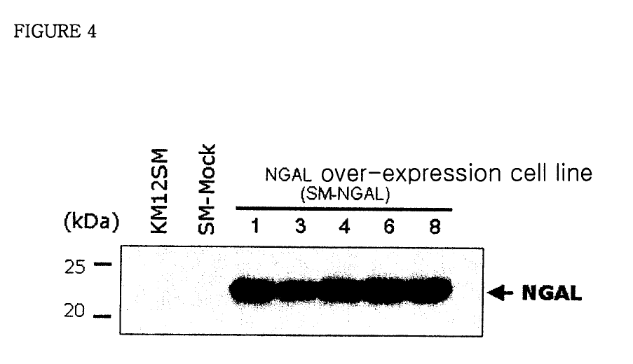

The isolated colorectal cancer clones were examined by Northern blot (FIG. 3) and Western blot (FIG. 4). FIG. 3 is a photograph of Northern blot analysis showing the over-expression of lipocalin 2 gene in the established lipocalin 2 over-expressing colorectal cancer cell line (SM-NGAL). FIG. 4 is a photograph of Western blot analysis showing the over-expression of lipocalin 2 protein in the concentrated culture supernatant of colorectal cancer cell lines (SM-NGAL #1, 3, 4, 6 and 8) confirmed to over-express lipocalin 2. Here in FIG. 3, SM-Mock is a control, in which only mock lentivirus was transduced into the colorectal cancer cell line. Band A indicates lipocalin 2 over-expressed by the recombinant lentivirus and band B indicates lipocalin 2 expressed endogenously in colorectal cancer cell line KM12SM.

As shown in FIG. 3 and FIG. 4, the level of lipocalin 2 expression was significantly higher in the lipocalin 2 over-expressing clones than in the control clones.

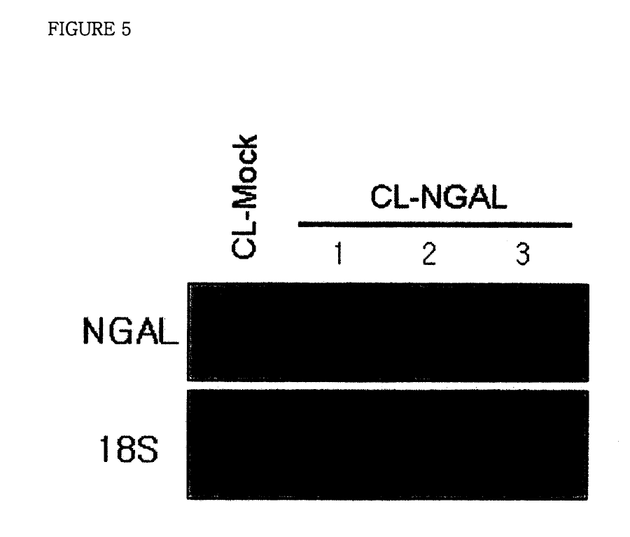

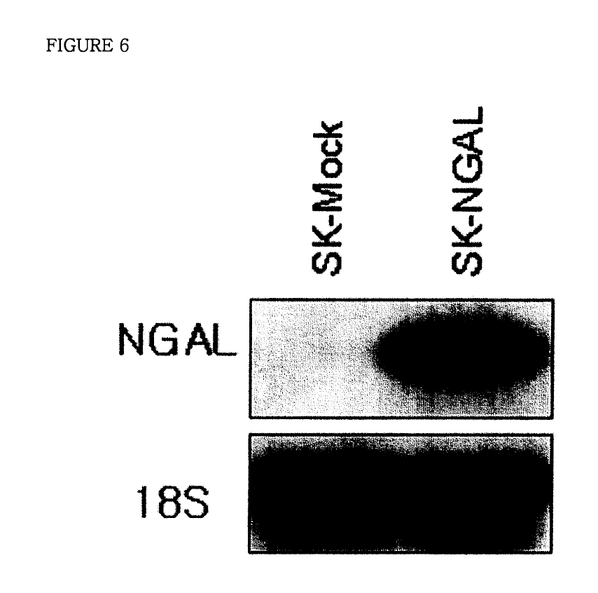

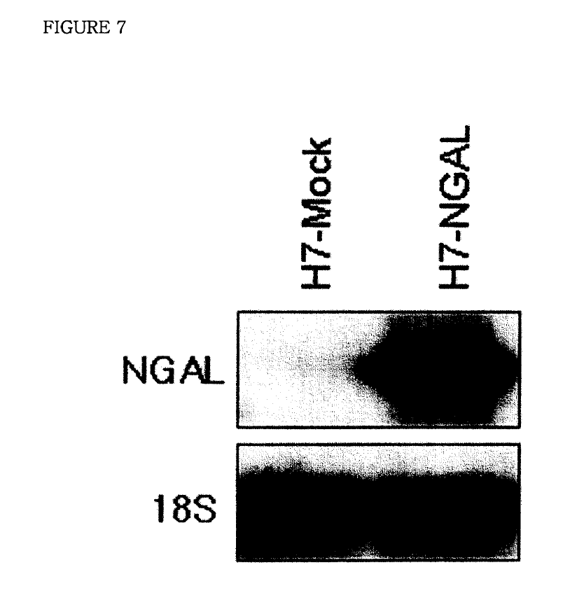

Individual clones of liver cancer cell lines Chang liver (FIG. 5), SK-Hep1 (FIG. 6) and Huh-7 (FIG. 7) were also examined by Northern blot analysis. FIG. 5-FIG. 7 are photographs of Northern blot analysis showing the over-expression of lipocalin 2 in each established liver cancer cell line (FIG. 5: Chang liver, FIG. 6: SK-Hep-1, FIG. 7: Huh-7).

As shown in FIGS. 5-7, the level of lipocalin 2 expression was significantly higher in the lipocalin 2 clones (FIG. 5: CL-NGAL, FIG. 6: SK-NGAL, FIG. 7: H7-NGAL) than in the control clones (FIG. 5: CL-Mock, FIG. 6: SK-Mock, FIG. 7: H7-Mock).

### Example 4

### Preparation of Recombinant Lipocalin 2 Protein

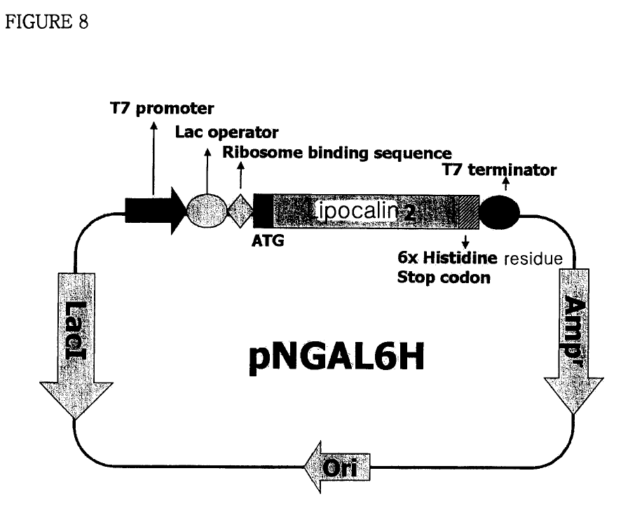

PCR was performed with primers of 5′-GGAATTCCATATGCAGGACTCCACCTCAGAC-3′ (SEQ. ID. No 9) and 5′-CGCGGATCCTCAATGGTGATGGTGATG-3′ (SEQ. ID. No 10) by using the lipocalin 2 expressing recombinant lentivirus expression vector (pLenti-NGAL) as a template to obtain a lipocalin 2 structural gene fragment. The obtained gene fragment contains a sequence region ranging from the 21^{st} amino acid to the 178^{th} amino acid (SEQ. ID. No 11) of the whole lipocalin 2 protein amino acid sequence which includes secretory signal sequence, and ATG start codon was added thereto for the expression of E. coli and so was 6 histidine codons at 3′-end for easy purification. The resultant lipocalin 2 gene PCR fragment was treated with Nde I/BamH I, which was inserted into the E. coli expression vector pET11a (Novagen, Germany). The constructed recombinant lipocalin 2 expression vector was named pNGAL6H (FIG. 8). FIG. 8 shows a cleavage map of pNGAL6H expression vector for the mass-expression of lipocalin 2. E. coli BL21(DE3) was transformed with the recombinant lipocalin 2 expression vector pNGAL6H, which was used for the expression of recombinant lipocalin 2 protein.

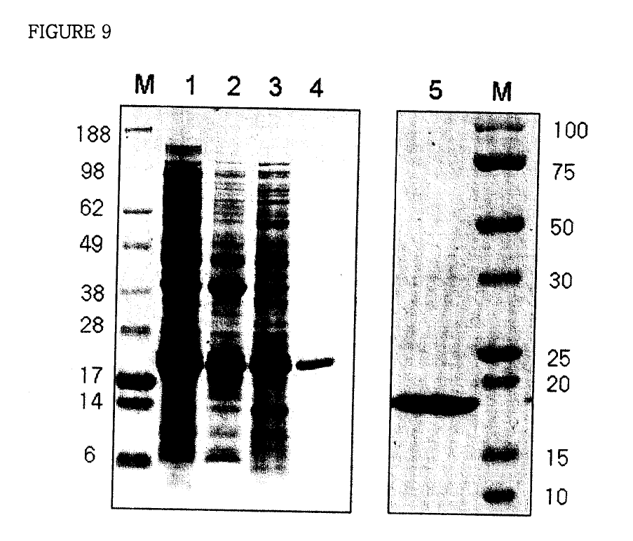

The recombinant lipocalin 2 expressing E. coli cells were cultured, harvested and homogenized to obtain water-soluble cell fractions. The fractions were then dialyzed against PBS. After dialysis, water-soluble fractions were re-separated and purified by affinity column chromatography using 6 histidine tag attached to C-terminal of lipocalmin 2. Lipocalin 2 protein was eluted by using 1 M imidazole, followed by dialysis using sodium phosphate buffer (pH 6.6). The recombinant lipocalin 2 protein was purified by FPLC column chromatography to enhance the purity of the protein. The final FPLC purification was performed in 20 mM sodium phosphate buffer (pH 6.6) using SP-Sepharose resin by 0-0.5 M NaCl density gradient. The expression of recombinant lipocalin 2 protein was confirmed by polyacrylamide gel electrophoresis (FIG. 9). FIG. 9 is a photograph of polyacrylamide gel illustrating the processes of lipocalin 2 expression in E. coli and purification thereof. Lane M is a molecular weight marker, lane 1 is the entire cell lysate, lane 2 is an inclusion body fraction, lane 3 is a water-soluble fraction, lane 4 is lipocalin 2 protein purified by histidine affinity column chromatography and lane 5 is the lipocalin 2 protein purified by FPLC.

As shown in FIG. 9, lipocalin 2 protein was confirmed to be expressed mainly as a water-soluble protein.

### Example 5

### Inhibitory Effect of Lipocalin 2 on the Invasion of a Colorectal Cancer Cell and a Liver Cancer Cell

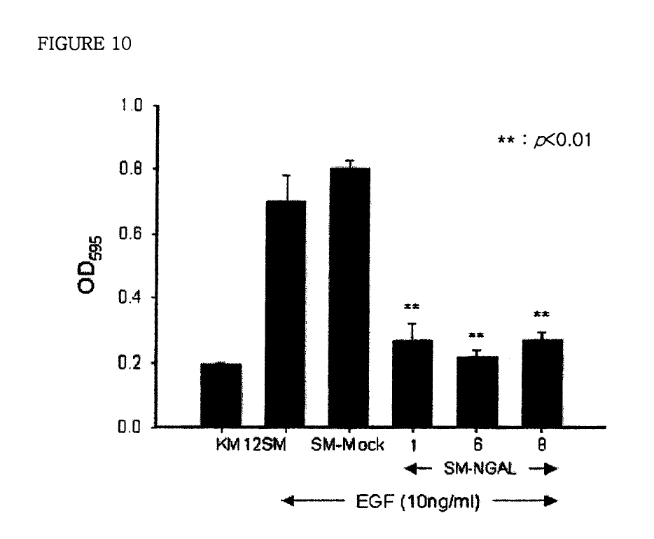

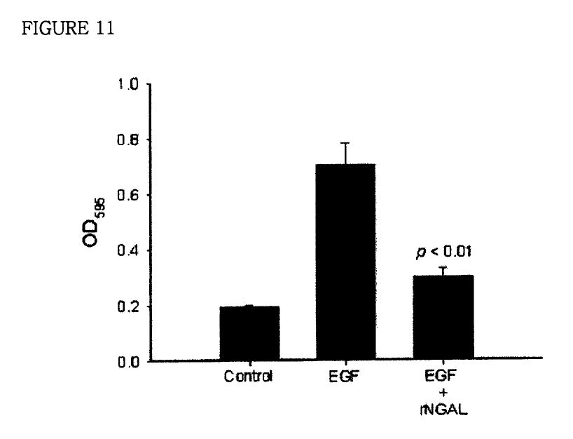

To investigate the inhibitory effect on the invasion of cancer cells of endogenous lipocalin 2 and exogenous recombinant lipocalin 2 protein (rNGAL), in vitro invasion assay was performed. First, the invasiveness of colorectal cancer cells was investigated using a transwell (Costar, USA) with a polycarbonate filter (pore size: 8 μm; diameter: 6.5 mm). Precisely, 40 μg of matrigel (BD Biosciences, USA) was distributed on the upper surface of the filter, on which 1×10⁵ cells were distributed. Then, culture medium containing 10 ng/ml of epidermal growth factor (EGF) was placed under surface of the filter, followed by culture for 2 days. The cells on the upper surface of the filter were removed by a cotton swab and the cells passed through to the under surface were stained with crystal violet, which were eluted in 30% acetic acid. Then, OD₅₉₅ was measured to determine the invasiveness (FIG. 10 and FIG. 11). FIG. 10 is a graph showing the invasiveness of human colorectal cancer cell line KM12SM when lipocalin 2 gene is over-expressed therein, and FIG. 11 is a graph showing the invasiveness of human colorectal cancer cell line KM12SM treated with the recombinant protein of lipocalin 2.

As shown in FIG. 10, the invasiveness was significantly reduced in lipocalin 2 over-expressing colorectal cancer cells (SM-NGAL; clones #1, #6, and #8), compared with control cells (KM-12SM and SM-Mock). As shown in FIG. 11, the invasiveness was also reduced in the cancer cells (EGF+rNGAL) treated with exogenous recombinant lipocalin 2 protein. The above results indicate that the lipocalin 2 reduces the invasiveness of colorectal cancer cells.

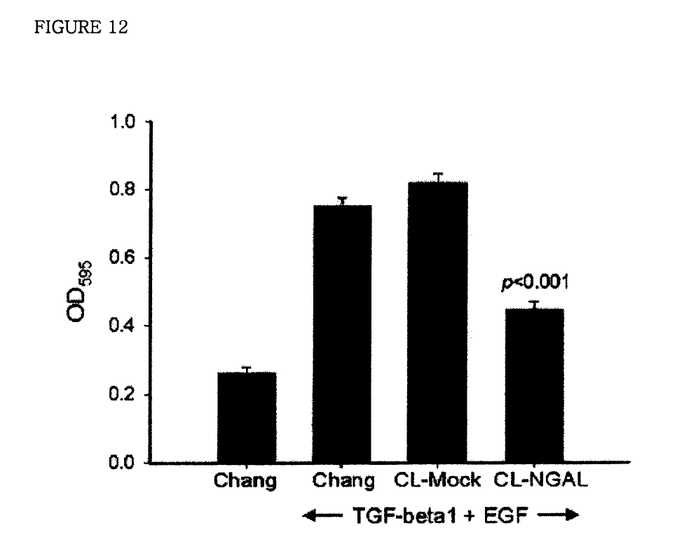

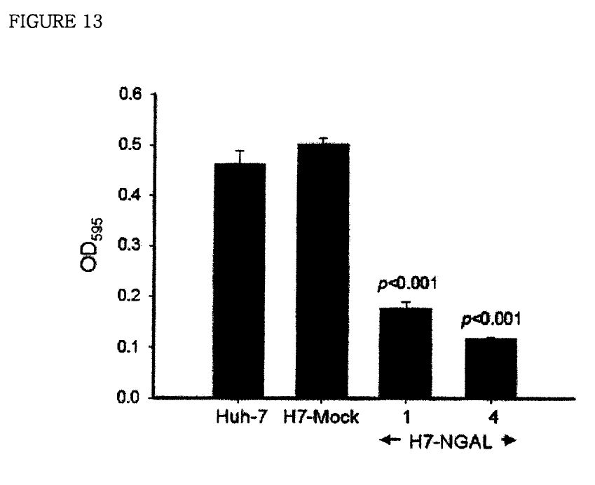

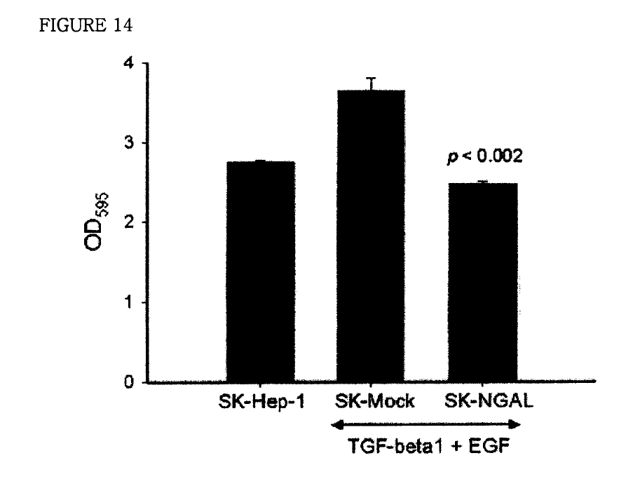

The inhibitory effect of lipocalin 2 on the invasion of liver cancer cells was also investigated by the same manner as described above. Precisely, 5×10⁴ liver cancer cells (Chang liver, Huh-7 or SK-Hep-1) were distributed on the upper face of a filter, with the lower face of the filter facing a medium containing 5 ng/ml of TGF-beta 1 and 10 ng/ml of EGF, followed by culture for 24 hours. The invasiveness was measured by the same manner as described above (FIG. 12-FIG. 14). FIG. 12-FIG. 14 are graphs showing the invasive capacities of liver cancer cell lines Chang liver (FIG. 12), Huh-7 (FIG. 13) and SK-Hep-1 (FIG. 14) when lipocalin 2 protein is over-expressed in them.

As shown in FIG. 12-FIG. 14, the invasiveness of lipocalin 2 over-expressing liver cancer cell lines, Chang liver (CL-NGAL), Huh-7 (H7-NGAL) and SK-Hep-1 (SK-NGAL), was much reduced, compared with that in the control, which was consistent with the above experimental results on colorectal cancer cell lines.

### Example 6

### Inhibition of MMP-2 Gene Expression in Liver Cancer Cells by the Over-Expression of Lipocalin 2

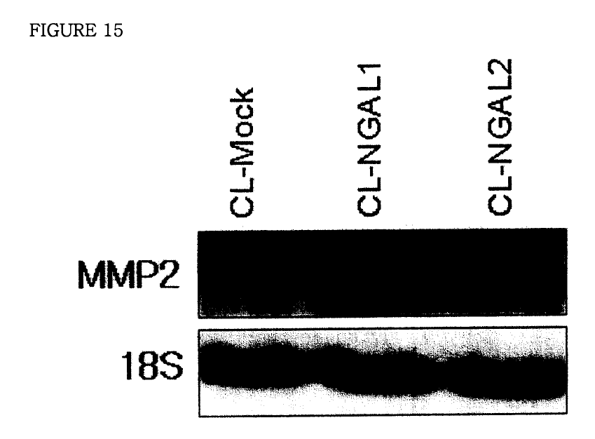

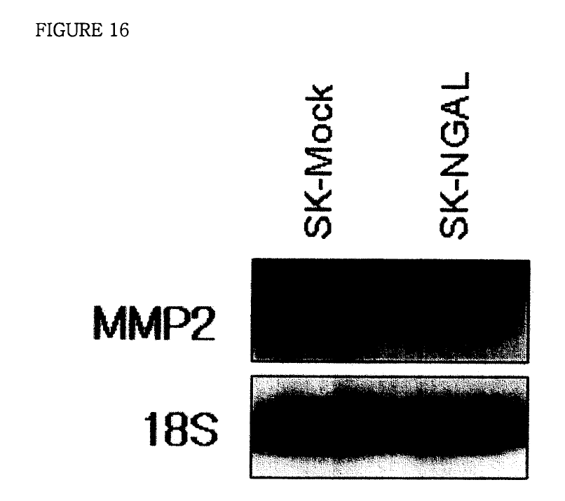

To investigate the effect of the over-expression of lipocalin 2 on the liver cancer cell lines Chang liver and SK-Hep-1, a control (mock) and lipocalin 2 over-expressing cell lines were prepared by using the recombinant lentivirus prepared in the above Example 2. 4-5 individual stable clone was selected from each recombinant cell line. mRNAs and proteins were examined by Northern blot and Western blot analysis. As a result, lipocalin 2 expression was not observed in control clones, while lipocalin 2 expression was highly detected in lipocalin 2 expressing cell lines. Northern blotting was also performed to investigate MMP-2 expressions in control and lipocalin 2-expressing clones of Chang liver and SK-Hep-1 (FIG. 15 and FIG. 16). FIG. 15 and FIG. 16 are photographs of Northern blot analysis illustrating that MMP-2 expression was reduced by lipocalin 2 in Chang liver and SK-Hep-1 cell lines.

As shown in FIG. 15, MMP-2 expression was remarkably reduced by lipocalin 2 expression in lipocalin 2 over-expressing Chang liver cell lines (CL-NGAL1, CL-NGAL2), compared with that in the control cell line (CL-Mock). As shown in FIG. 16, MMP-2 expression was also remarkably reduced by lipocalin 2 expression in lipocalin 2 over-expressing liver cancer cell line SK-Hep-1 (SK-NGAL) compared with that in a control cell line (SK-Mock).

### Example 7

### Inhibition of MMP-2 Expression by the Recombinant Lipocalin 2 Protein in Liver Cancer Cells

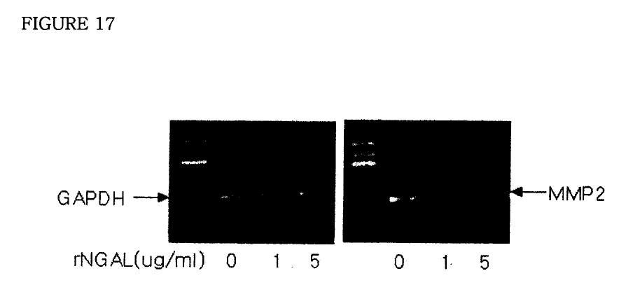

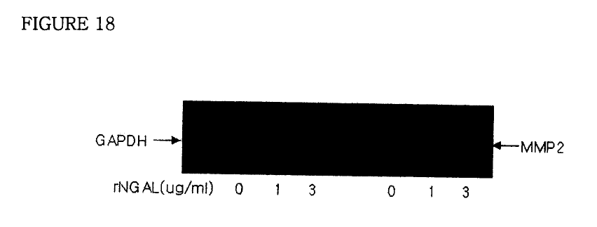

Chang liver cells were cultured upto approximately 90% density on a 100-mm culture dish in DMEM supplemented with 10% FBS. The cells were treated with the recombinant lipocalin 2 protein, prepared from E. coli as described in the above Example 4, in the same medium for 6 hours by the concentrations of 0, 1, or 5 μg/ml. The cells were also treated with the recombinant lipocalin 2 protein in the serum-free medium by the concentrations of 0, 1, or 3 μg/ml. Then, RT-PCR was performed to investigate MMR-2 expressions in Chang liver cancer cells at different concentrations of the recombinant lipocalin 2 protein (FIG. 17 and FIG. 18). FIG. 17 and FIG. 18 are agarose gel photographs of RT-PCR products showing the expressions of MMP-2 gene in liver cancer cell line Chang liver treated with the recombinant lipocalin 2 protein in a 10% FBS containing medium (FIG. 17) and in a serum-free medium (FIG. 18).

As shown in FIG. 17, MMP-2 expression was decreased by the treatment of 1 μg/ml of the recombinant lipocalin 2 protein in a medium supplemented with 10% FBS, compared with that in the control. A house-keeping gene GAPDH was quantified to confirm that the experiment was performed with the equal amount of the test sample. As shown in FIG. 18, MMP-2 expression in Chang liver cells was also decreased by the treatment of 1 μg/ml of lipocalin 2 protein in the serum-free DMEM.

### Example 8

### The Effect of the Over-Expression of Lipocalin 2 on Cancer Cell Proliferation and Solid Tumor Growth

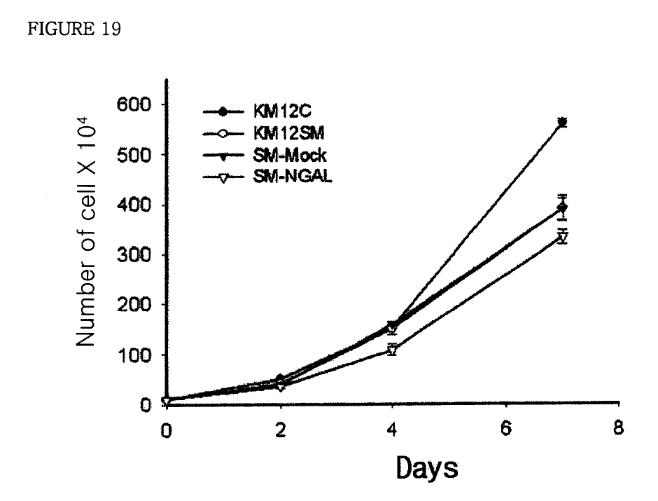

Following experiments were performed to investigate the effect of the over-expression of lipocalin 2 on cancer cell proliferation and solid tumor growth. First, the effect of lipocalin 2 over-expression on cancer cell proliferation was examined, for which colorectal cancer cell line KM12SM was inoculated on a 6-well culture plate at the concentration of 1×10⁵ cells per well. The viable cells were counted every 2-3 days after staining with trypan blue (FIG. 19). FIG. 19 is a graph showing the numbers of cells measured by cell proliferation assay to investigate the effect of the over-expression of lipocalin 2 on the colorectal cancer cell growth.

As shown in FIG. 19, the proliferation rate of cancer cells in lipocalin 2 over-expressing KM12SM cell line (SM-NGAL) was slightly decreased, compared with those in controls (KM12C, KM12SM and SM-Mock), though it was not statistically significant.

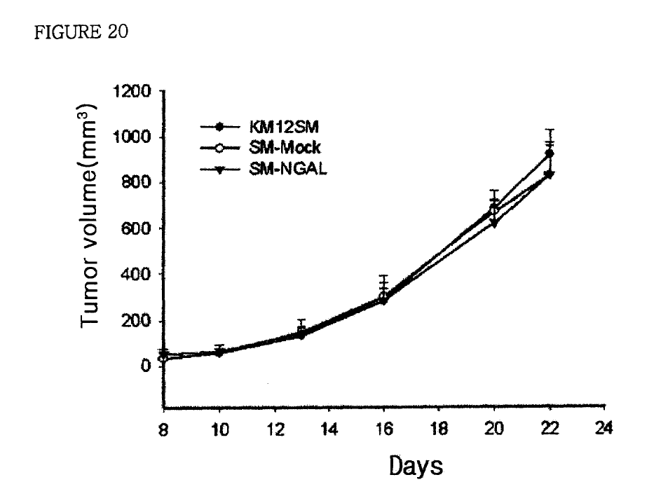

To investigate the in vivo effect of lipocalin 2 on tumor growth, control cells (KM12SM and SM-Mock) and lipocalin 2 over-expressing colorectal cancer cells (SM-NGAL) were injected subcutaneously to nude mice by 2×10⁶ cells per mouse. The size of a solid tumor was measured every 3-4 days over 24 days (FIG. 20). FIG. 20 is a graph illustrating the tumor growths of lipocalin 2 over-expressing colorectal cancer cells and control cells which were subcutaneously injected into mice.

As shown in FIG. 20, there was no significant difference in the growth of a solid tumor derived from lipocalin 2 over-expressing KM12SM colorectal cancer cells (SM-NGAL) and control cells (KM12SM and SM-Mock).

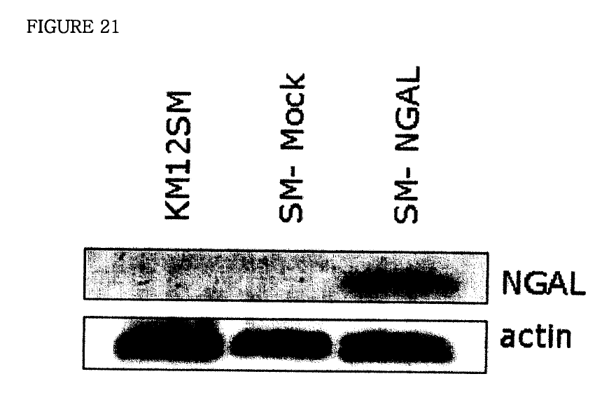

To avoid the possibility that the similar tumor growth in the experimental group and the control group might be attributed to suppression of lipocalin 2 expression in tumor tissues, proteins extracted from tumor tissues were examined by Western blot analysis (FIG. 21). FIG. 21 is a photograph of Western blot analysis of total proteins extracted from the solid tumor tissues illustrating that lipocalin 2 was expressed in the colorectal cancer growing under the skin of a mouse.

As shown in FIG. 21, the lipocalin 2 was expressed stably through all the experimental period in tumors derived from SM-NGAL cell line. Therefore, it was proved that the over-expression of lipocalin 2 does not affect cancer cell proliferation and solid tumor growth in general.

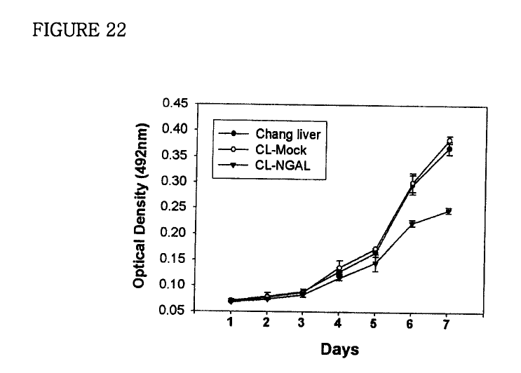

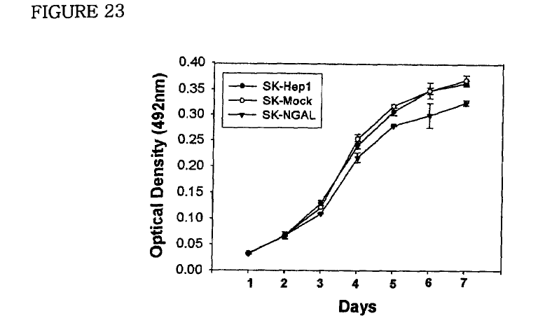

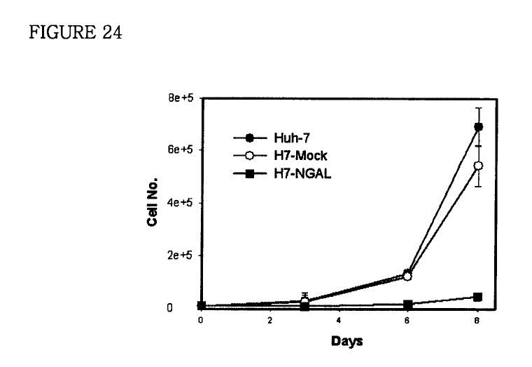

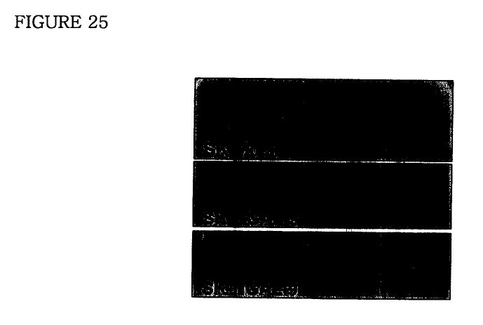

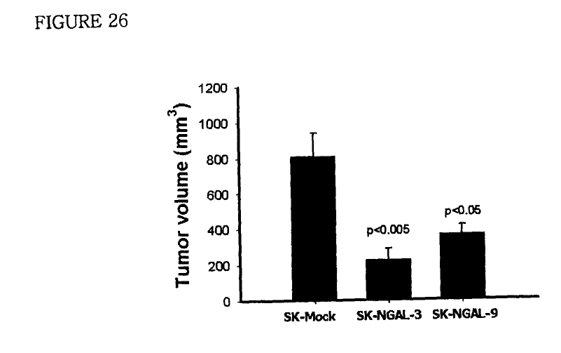

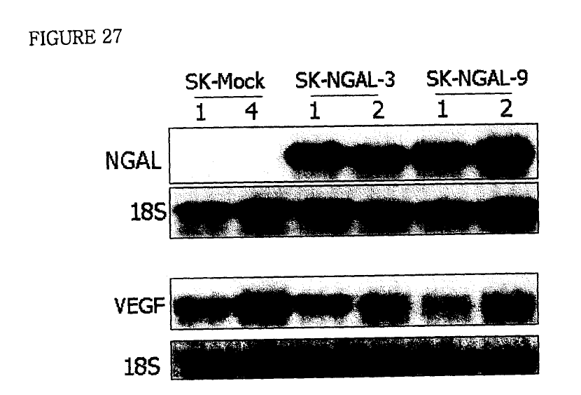

However, it is strongly believed that lipocalin 2 has a tumor cell-specific growth inhibitory effect. To investigate the effect of the over-expression of lipocalin 2 on the proliferation of liver cancer cells, 1×10⁴ cells of control liver cancer cell lines (Chang liver, SK-Hep-1 and Huh-7) and lipocalin 2 over-expressing liver cancer cell lines (CL-NGAL, SK-NGAL and H7-NGAL) were inoculated on culture dishes. Then, cells were collected every 2-3 days, and viable cells were counted by using trypan blue staining or using CellTiter 96 Aqueous Non-Radioactive cell proliferation assay kit (Promega, USA) (FIG. 22-FIG. 24). As a result, unlike in colorectal cancer cell lines, the over-expression of lipocalin 2 reduced the proliferation of liver cancer cells significantly (FIG. 22-FIG. 24). Moreover, 1×10⁷ control cells (SK-Mock) and lipocalin 2 over-expressing liver cancer cells (SK-NGAL) were subcutaneously injected to BALB/c nude mice and the tumor growth was observed. As a result, the size of the tumors derived from lipocalin 2 over-expressing SK-NGAL cells was remarkably decreased, compared with that of the control (FIG. 25 and FIG. 26). FIG. 25 is a photograph showing the tumors formed under the skin of the nude mice with the injection of the control cells (SK-Mock) and lipocalin 2 over-expressing liver cancer cells (SK-NGAL-3; SK-NGAL-9), and FIG. 26 is a graph showing the values representing the volumes of each tumor of experimental groups. Based on the finding that the in vivo tumor growth inhibitory effect was much greater than in vitro tumor growth inhibitory effect, it was suggested that lipocalin 2 might have an inhibitory effect on angiogenesis, a key factor for tumor growth, in addition to the tumor growth inhibitory effect. Vascular Endothelial cell Growth Factor (VEGF) is an essential factor for angiogenesis in a tumor and the inhibition of VEGF expression leads to the suppression of a tumor growth. To investigate the effect of the over-expression of lipocalin 2 on VEGF expression, RNAs were extracted from control and lipocalin 2 over-expressing liver tumors formed in the nude mice, followed by Northern blot analysis to measure the levels of VEGF and lipocalin 2 mRNA (FIG. 27). As a result, VEGF expression was significantly decreased in the tumor derived from lipocalin 2 over-expressing liver cancer cells, compared with the control tumor. FIG. 27 is a photograph of Northern blot analysis investigating the levels of lipocalin 2 and VEGF mRNA by using RNAs extracted from tumors (two per group) formed under the skin of the nude mice with the injection of control (SK-Mock) and lipocalin 2 over-expressing liver cancer cells (SK-NGAL-3; SK-NGAL-9). From the above results, it was confirmed that lipocalin 2 not only inhibits the proliferation of liver cancer cells but also interrupts angiogenesis in a tumor tissue, leading to the inhibition of tumor growth.

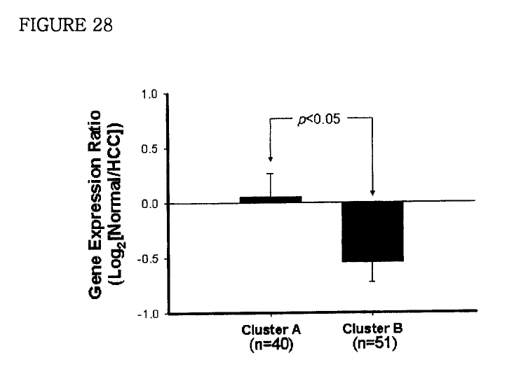

Through inhibition of cell proliferation, invasion, and angiogenesis, which seems to be mediated by suppression of MMP-2 expression and VEGF expression, lipocalin 2 may inhibit the tumor growth and progression in liver cancers. Thus, the expression of lipocalin 2 might contribute to good prognosis of a liver cancer patient. To investigate the relationship between lipocalin 2 expression and prognosis of liver cancer patients, microarray results of liver cancer patients deposited on the public database (Gene Expression Omnibus, National Center for Biological Information, USA; www.ncbi.nlm.nih.gov/geo/; human microarray platform, GPL1528; human HCC microarray data, GSE1898; Lee et al., Hepatology 40:667-676, 2004) were analyzed. As a result, the ratio of lipocalin 2 expressed in liver cancer tissues to that expressed in normal liver tissues (Log2[Normal/HCC]) was significantly reduced (p<0.05) in a patient group with good prognosis (cluster B), compared with the ratio in a patient group with poor prognosis (cluster A). These results indicate that the level of lipocalin 2 in a liver cancer patient can be used as a standard index to predict a prognosis of a liver cancer patient (FIG. 28).

### Example 9

### Inhibition of Colorectal Cancer Liver Metastasis by Lipocalin 2

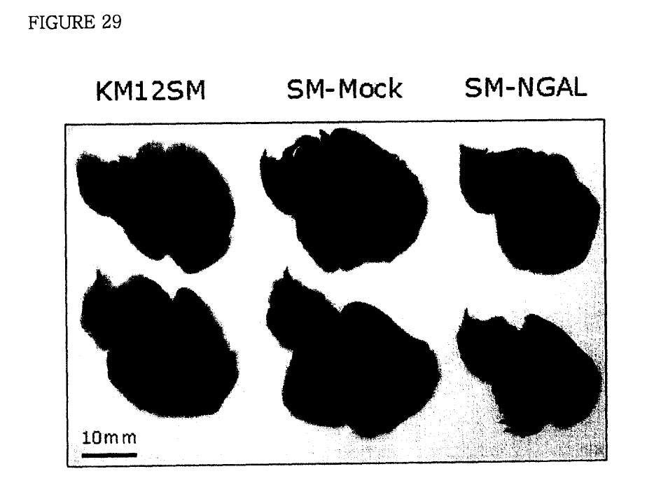

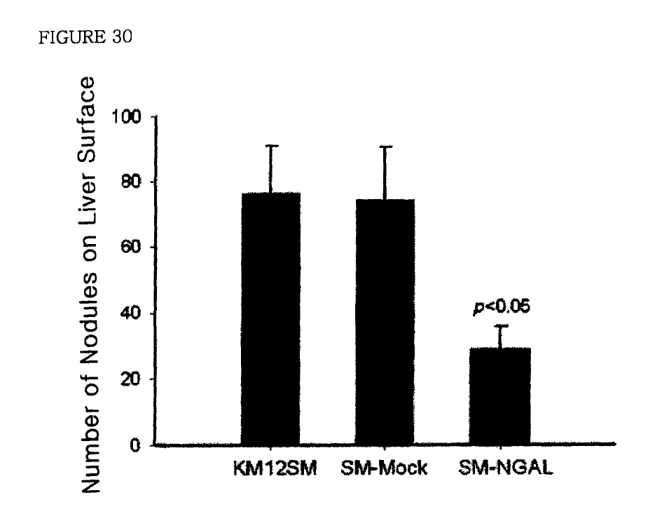

Control cells (KM12SM and SM-Mock) and lipocalin 2 over-expressing colorectal cancer cells (SM-NGAL) were injected into spleens of random-bred male BALB/c nu/nu nude mice (Charles River Japan Inc., Japan) by 1×10⁶ cells per mouse, and then liver metastasis was observed. On the 21^{st} day after cell injection, mice were sacrificed and livers were taken out. Metastatic colonies in livers were measured (FIG. 29 and FIG. 30). FIG. 29 is a photograph showing the livers collected on the 21^{st} day after the injection of control cells (KM12SM and SM-Mock) and lipocalin 2 over-expressing colorectal cancer cells (SM-NGAL) into spleens of mice to induce liver metastasis. FIG. 30 is a graph showing the numbers of liver metastatic colonies found on the surfaces of the livers shown in FIG. 16a.

As shown in FIG. 29 and FIG. 30, the numbers of the metastatic colonies were approximately 60% decreased in the experimental group of mice bearing tumors derived from lipocalin 2 over-expressing colorectal cancer cells (SM-NGAL), compared with those in the control group of mice bearing tumors derived from control cells (KM12SM and SM-Mock).

### Example 10

### Inhibition of Liver Metastasis of Colorectal Cancer Cells by the Recombinant Lipocalin 2 Protein

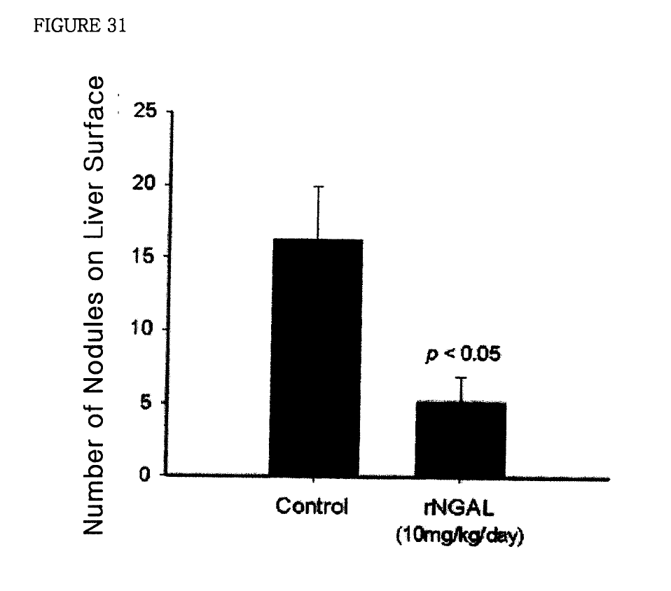

To investigate the inhibitory effect on the colorectal cancer liver metastasis by the treatment of exogenous recombinant lipocalin 2 protein, the metastasis inhibition test was performed using the animal models for liver metastasis that were established as described above. Human colorectal cancer cell line LS174T (American Type Culture Collection, USA) was injected into the spleens of a nude mice by 3×10⁵ cells per mouse, then the recombinant lipocalin 2 protein was injected intraperitoneally to the mouse everyday for 17 days by 10 mg/kg per mouse. On the 18^{th} day after the cell injection, mice were sacrificed and livers were collected. The numbers of metastatic colonies (derived from colorectal cancer) found on the surface of the liver were counted (FIG. 31). FIG. 31 is a graph showing the numbers of colorectal cancer colonies formed on the surface of the liver measured to examine the liver metastasis inhibitory effect of the recombinant lipocalin 2 protein.

As shown in FIG. 31, the numbers of metastatic colonies were at least about 60% reduced in an experimental group treated with the recombinant lipocalin 2 protein (rNGAL), compared with that in a control group.

### INDUSTRIAL APPLICABILITY

The pharmaceutical composition of the present invention inhibits cancer metastasis specifically, so that it can improve the effect of cancer treatment dramatically. And the diagnostic kit of the present invention and the method for the selection of a cancer metastasis risk group using the diagnostic kit of the invention enable the effective selection of a metastasis risk group by investigating lipocalin 2 expression in tumor tissues or body fluid, so that they are useful for the clinical treatment of a cancer patient. In addition, the pharmaceutical composition of the invention also inhibits the liver cancer cell proliferation and a solid tumor growth as well as the expression of VEGF, so that it can be effectively used for the treatment of liver cancer.

### SEQUENCE LIST TEXT

The SEQ. ID. No 1 is a nucleotide sequence of a whole human lipocalin 2 gene,

The SEQ. ID. No 2 is an amino acid sequence of a whole human lipocalin 2 protein,

The SEQ. ID. No 3 is a forward primer sequence for the whole human lipocalin 2,

The SEQ. ID. No 4 is a reverse primer sequence for the whole human lipocalin 2,

The SEQ. ID. No 5 is a forward primer sequence for pT-NGAL,

The SEQ. ID. No 6 is a reverse primer sequence for pT-NGAL,

The SEQ. ID. No 7 is a forward primer sequence for pLenti6/V5-D-TOPO,

The SEQ. ID. No 8 is a reverse primer sequence for pLEnti6/V5-D-TOPO,

The SEQ. ID. No 9 is a forward primer sequence for pLenti-NGAL,

The SEQ. ID. No 10 is a reverse primer sequence for pLenti-NGAL,

The SEQ. ID. No 11 is an amino acid sequence of matured human lipocalin 2 protein without secretory signal sequence.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A pharmaceutical composition for the inhibition of cancer growth and metastasis, which contains a gene encoding lipocalin 2 protein, an expression vector containing the gene or a mammalian cell transfected with the expression vector as effective ingredients.

2. The pharmaceutical composition according to claim 1, wherein the gene is represented by SEQ. ID. No 1.

3. The pharmaceutical composition according to claim 1, wherein the expression vector is a non-viral vector or a viral vector.

4. The pharmaceutical composition according to claim 3, wherein the viral vector is selected from the group consisting of adenovirus vector, retrovirus vector including lentivirus vector, adeno-associate virus vector or herpes simplex virus vector.

5. The pharmaceutical composition according to claim 4, wherein the lentivirus vector is pLenti-NGAL presented in FIG. 2.

6. The pharmaceutical composition according to any one of claims 1-5 further comprising a pharmaceutically acceptable carrier.

7. The pharmaceutical composition according to claim 1, which inhibits metastasis of colorectal cancer or liver cancer.

8. The pharmaceutical composition according to claim 1, which inhibits growth of liver cancer.

9. A pharmaceutical composition for the inhibition of cancer growth and metastasis containing a lipocalin 2 protein as an effective ingredient.

10. The pharmaceutical composition according to claim 9, wherein the lipocalin 2 protein is represented by SEQ. ID. No 2 or SEQ. ID. No 11.

11. The pharmaceutical composition according to claim 9 of claim 10, further comprising a pharmaceutically acceptable carrier.

12. The pharmaceutical composition according to claim 9, which inhibits metastasis of colorectal cancer or liver cancer.

13. The pharmaceutical composition according to claim 9, which inhibits growth of liver cancer.

14. A lentivirus expression vector for lipocalin 2, which is pLenti-NGAL presented in FIG. 2.

15. A recombinant lentivirus prepared by the transfection with the expression vector of claim 14.

16. A cell line transduced with the recombinant lentivirus of claim 15.

17. The cell line according to claim 16, which is a cancer cell line or a normal cell line.

18. The cell line according to claim 17, wherein the cancer cell line is selected from the group consisting of KM12C, SW480, KM12SM, SW620, Chang liver, SK-Hep-1 and Huh-7.

19. A method for the inhibition of cancer metastasis containing the step of administration of a pharmaceutical composition of claim 1 or claim 9 to a cancer patient.

20. The method for the inhibition of cancer metastasis according to claim 19, wherein the cancer is colorectal cancer or liver cancer.

21. A method for the inhibition of cancer growth containing the step of administration of a pharmaceutical composition of claim 1 or claim 9 to a cancer patient.

22. The method for the inhibition of cancer growth according to claim 21, wherein the cancer is liver cancer.

23. A diagnostic kit for the prediction of cancer metastasis, which contains a lipocalin 2-specific antibody or a set of primers which are specific to a gene encoding the lipocalin 2 protein.

24. The diagnostic kit for the prediction of cancer metastasis according to claim 23, wherein the antibody is a monoclonal antibody or a polyclonal antibody.

25. The diagnostic kit for the prediction of cancer metastasis according to claim 23, wherein the set of primers are represented by SEQ. ID. No 3 and SEQ. ID. No 4.

26. A method for the selection of a metastasis risk group using the kit of claim 23, which comprises the following steps:
i) obtaining a cancer sample from a cancer patient;
ii) preparing a sample for the detection of lipocalin 2 protein or mRNA encoding the protein by using a dissection sample, lysate and total RNA from the cancer sample;
iii) detecting lipocalin 2 protein or mRNA encoding the protein from the sample; and
iv) analyzing the level of the independent expression of lipocalin 2 or the combined expression level of lipocalin 2 and other metastasis suppressor genes.

27. The method for the selection of a metastasis risk group according to claim 26, wherein the step iii) is performed by selecting from a group consisting of in situ hybridization, immunohistochemical staining, ELISA (enzyme-linked immunosorbent assay), Western blot, Northern blot, RT-PCR and real-time RT-PCR.


SEQUENCE LISTING (11 sequences)

SEQ ID NO: 1 - DNA, 597 bp, Homo sapiens
atgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctga

SEQ ID NO: 2 - protein, 198 aa, Homo sapiens
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 3 - DNA, 28 bp, Artificial
  forward primer of lipocalin 2
caccatgcccctaggtctcctgtggctg

SEQ ID NO: 4 - DNA, 18 bp, Artificial
  backward primer of lipocalin2
tcagccgtcgatacactg

SEQ ID NO: 5 - DNA, 24 bp, Artificial
  forward primer of pTNGAL
atttaggtgacactatagaatact

SEQ ID NO: 6 - DNA, 45 bp, Artificial
  backward primer of lipocalin 2 containing 6 His and SacII restriction site
tccccgcggtcaatggtgatggtgatgatggccgtcgatacactg

SEQ ID NO: 7 - DNA, 21 bp, Artificial
  forward sequencing primer of lipocalin 2
cgcaaatgggcggtaggcgtg

SEQ ID NO: 8 - DNA, 21 bp, Artificial
  backward sequencing primer of lipocalin 2
accgaggagagggttagggat

SEQ ID NO: 9 - DNA, 31 bp, Artificial
  forward primer of lipocalin 2 containing NdeI restriction site
ggaattccatatgcaggactccacctcagac

SEQ ID NO: 10 - DNA, 30 bp, Artificial
  backward primer of lipocalin 2 containing BamHI restrction site
cgcggatcctcaatggtgatggtgatgatg

SEQ ID NO: 11 - protein, 178 aa, Homo sapiens
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG